(19) United States
(12) Patent Application Publication
(10) Pub. No.: US 2004/0255956 A1
(43) Pub. Date: Dec. 23, 2004

(54) Post-conditioning for the reduction of ischemic-reperfusion injury in the heart and other organs

(76) Inventors: Jakob Vinten-Johansen, Grayson, GA (US); Zhi-Qing Zhao, Norcross, GA (US)

Correspondence Address: NEEDLE & ROSENBERG, P.C., SUITE 1000, 999 PEACHTREE STREET, ATLANTA, GA, 30309-3915, US

(21) Appl. No.: 10/499,052

(22) PCT Filed: Dec. 20, 2002
(86) PCT No.: PCT/US02/41354

Related U.S. Application Data
(60) Provisional application No. 60/343,275, filed on Dec. 21, 2001.

(51) Int. Cl. A61B 17/12

(52) U.S. Cl. 128/898

(57) ABSTRACT

The present invention provides a method of post-conditioning reperfusion of an organ or tissue injured by ischemia. Also provided is a method of treating a myocardial infarction in a subject to prevent injury to the heart following reperfusion of the heart.

This application claims priority to U.S. Provisional Application No. 60/343,275, filed Dec. 21, 2001, which is incorporated by this reference in its entirety.

### BACKGROUND OF THE INVENTION

1. Field of the Invention

The present invention relates to the treatment of organs and tissues injured by ischemia. Specifically, the present invention relates to preventing reperfusion injury in organs and tissues that have suffered an ischemic event.

2. Background Art

Heart disease is the leading cause of premature, permanent disability among American workers, accounting for nearly 20 percent of Social Security disability payments. About 20 million Americans live with the effects of heart disease, and over six million people have heart attacks each year. Every year nearly 50% of patients suffering first-time heart attacks die from myocardial infarctions.

The heart needs a constant and uninterrupted blood supply for normal and continued function. When a patient has a heart attack, the blood flow to part of the heart is stopped, resulting in ischemia. The heart will lose its functional capabilities, and the ischemic part of the heart is in jeopardy of dying, resulting in focal necrosis of the heart tissue. A heart attack can be treated either by percutaneous transluminal coronary angioplasty (PTCA) or by a more invasive procedure, coronary artery bypass graft surgery (CABG). Both procedures can open up a blocked blood vessel (coronary artery) to restore blood supply to the heart muscle, a process called reperfusion. Although the beneficial effects of early reperfusion of ischemic myocardium with thrombolytic therapy, PTCA, or CABG are now well established, an increasing number of studies indicates that reperfusion also induces an additional injury to ischemic heart muscle, such as the extension of myocardial necrosis, i.e., extended infarct size and impaired contractile function and metabolism. Reperfusion injury can extend not only acutely, but also over several days following the heart attack.

Over the last two decades, numerous efforts have been made to find a therapy that could limit reperfusion-induced extension of infarct size following a heart attack. Many studies have focused on targeting and exploring some pharmacological agents in an attempt to reduce infarct size, with the ultimate clinical aim of reducing post-infarct morbidity and mortality, and improving the patient's lifestyle and longevity. In the long run, however, results in reducing infarct size have been rather unsatisfactory. It is generally accepted that drugs tested in pre-clinical studies may have the ability to delay the appearance of myocardial injury, but fail to permanently produce a true reduction in infarct size.

The present invention provides a method of treatment (post-conditioning) in which reperfusion injury to an organ or tissue already undergoing total or subtotal ischemia can be significantly reduced by modifying the perfusion (blood flow) conditions during onset of reperfusion, i.e., modification of the early reperfusion period.

### SUMMARY OF THE INVENTION

Provided herein is a method of preventing injury to an organ or tissue in a subject during or after reperfusion following an ischemic event to the organ or tissue, comprising: a) stopping perfusion of the organ or tissue for from about 5 seconds to about 5 minutes; b) perfusing the organ or tissue for from about 5 seconds to about 5 minutes; c) repeating steps a) and b) sequentially for from about 2 to about 50 times; and d) ending stopping perfusion of the organ or tissue, thereby preventing injury to the organ or tissue in the subject following an ischemic event.

Also provided is a method of preventing injury to a heart in a subject diagnosed with an ischemic event of the heart, comprising: a) clearing a lumen of a coronary artery; b) perfusing the heart for from about 5 seconds to about 5 minutes; c) stopping perfusion of the heart for from about 5 seconds to about 5 minutes; d) repeating steps b) and c) sequentially for from about 2 to about 50 times; and e) resuming perfusion of the heart, thereby preventing injury to the heart in the subject diagnosed with an ischemic event of the heart.

Provided herein is a method of preventing injury to an organ or tissue in a subject during or after reperfusion following an ischemic event to the organ or tissue, comprising: a) reducing perfusion of the organ or tissue for from about 5 seconds to about 5 minutes; b) perfusing the organ or tissue for from about 5 seconds to about 5 minutes; c) repeating steps a) and b) sequentially for from about 2 to about 50 times; and d) ending stopping perfusion of the organ or tissue, thereby preventing injury to the organ or tissue in the subject following an ischemic event.

Also provided is a method of preventing injury to a heart in a subject diagnosed with an ischemic event of the heart, comprising: a) clearing a lumen of a coronary artery; b) perfusing the heart for from about 5 seconds to about 5 minutes; c) reducing perfusion of the heart for from about 5 seconds to about 5 minutes; d) repeating steps b) and c) sequentially for from about 2 to about 50 times; and e) resuming perfusion of the heart, thereby preventing injury to the heart in the subject diagnosed with an ischemic event of the heart.

### BRIEF DESCRIPTION OF THE DRAWINGS

FIG. 1 shows the experimental protocol used to determine the effect of one possible variation in post-conditioning on myocardium after ischemia (I) and reperfusion (R). Control group (n=10); Post-con (n=10); Pre-con (n=9): Ischemic preconditioning was elicited by 5 minutes of coronary occlusion followed by 10 minutes of reperfusion before 60 minutes of left anterior descending coronary artery (LAD) occlusion, and post-conditioning 3 cycles of 30 seconds of reperfusion followed by 30 seconds of occlusion before 3 hours of reperfusion, respectively. Post-con is post-conditioning; pre-con is pre-conditioning.

FIG. 2 is a bar graph showing a reduction in myocardial infarction size by ischemic post-conditioning as determined by triphenyltetrazolium chloride (TTC) vs. pre-conditioning staining. Area at risk (AAR) relative to left ventricular (LV) mass (AAR/LV) and area of necrosis (AN) expressed as a percentage of AAR (AN/AAR). Ischemic post-conditioning significantly reduced AN/AAR by 48% compared with Control group, and therefore demonstrated equipotent cardioprotection to that of ischemic preconditioning, *P<0.05 vs. Control group. Values are group mean±S.E.M.

FIG. 3 is a bar graph showing a reduction in myocardial edema in the LAD-perfused myocardium by ischemic post-conditioning. Normal: non-ischemic zone; Isch-epi: ischemic subepicardium; Isch-endo: ischemic subendocardium. Ischemic post-conditioning significantly reduced tissue water content compared with Control group. *P<0.05 vs. normal zone. †P<0.01 vs. Control group. Values are group mean±S.E.M.

FIG. 4 is a graph showing the plasma creatine kinase (CK) activity during the course of coronary occlusion and reperfusion. Plasma CK activity was comparable between the two groups at baseline and after ischemia. Consistent with reduction in infarction size, ischemic post-conditioning significantly decreased CK activity starting at 2 hours of reperfusion relative to the Control group values. Values are mean±S.E.M.; *P<0.01 vs. Baseline and Isch values. †p<0.05 vs. Control group.

FIG. 5 is a line graph showing regional transmural myocardial blood flow in the ischemic-reperfused myocardium. Values at baseline and during ischemia were comparable between the two groups. Hyperemia at 15 minutes of reperfusion was significantly inhibited by ischemic pre- and post-conditioning. Values are mean±S.E.M. *P<0.05 vs. ischemia †P<0.05 vs. Control group.

FIG. 6 is a line graph showing post-ischemic-reperfusion endothelium function of non-ischemic left circumflex coronary artery (LCX) coronary artery rings and ischemic-reperfused (LAD) coronary artery rings assessed as responses to incremental concentrations of acetylcholine in organ chambers. Responses to acetylcholine at reperfusion were significantly blunted vs. responses of the non-ischemic LCX coronary artery rings. Response in ischemic post-conditioning was significantly increased, suggesting better endothelial function and avoidance of ischemic-reperfusion injury with post-conditioning. Values are Mean±S.E.M. of at least 12 rings from 5 dogs. *P<0.05 LAD in Control group vs. ischemic post- and pre-conditioning.

FIG. 7 is a line graph showing responses of non-ischemic LCX coronary rings and ischemic-reperfused (LAD) coronary rings to the vascular smooth muscle vasodilator, nitroprusside. No group difference was detected in all groups, suggesting that vascular smooth muscle function was normal and comparable among groups.

FIG. 8 is a bar graph showing the inhibition in adherence of unstimulated fluorescence-labeled neutrophils to coronary endothelium by ischemic post-conditioning vs. pre-conditioning. The degree of adherence correlates with the degree of damage sustained by the coronary artery endothelium, related to loss of basal generation of nitric oxide or adenosine. LCX: non-ischemic left circumflex coronary artery; LAD: ischemic/reperfused left anterior descending coronary artery; Post-LAD: LAD in ischemic post-conditioning group; Pre-LAD: LAD in ischemic pre-conditioning group. As potent as the protection by ischemic preconditioning, ischemic post-conditioning significantly inhibited neutrophil adherence to coronary endothelium compared with Control group. Values are group mean±S.E.M. *P<0.05 vs. LCX; †P<0.01 vs. LAD in Control group.

FIG. 9 shows tissue myeloperoxidase (MPO in delta absorbance A units/minute, (abs/min.)) activity as a marker of neutrophil accumulation in non-ischemic (Normal) and ischemic zones in the different experimental groups after LAD ischemia and reperfusion. Increased MPO activity was seen at the end of reperfusion in the control AAR. Ischemic post-conditioning significantly decreased MPO activity compared with Control group, and was comparable to that in the preconditioning group. Bar height represents mean±SEM. *p<0.05 vs. normal tissue; †p<0.05 Post-con and Pre-con group vs. Control group.

### DETAILED DESCRIPTION OF THE INVENTION

It must be noted that, as used in the specification and the appended claims, the singular forms “a,” “an,” and “the” include plural referents unless the context clearly dictates otherwise. Thus, for example, reference to “an agent” includes multiple copies of the agent and can also include more than one particular species of agent.

Provided herein is a method of minimizing damage in an ischemic/reperfused heart muscle by providing a protective effect when it is applied in the treatment of ischemic heart disease in conjunction with percutaneous transluminal coronary angioplasty (PTCA) and/or coronary artery bypass grafting surgery (CABG). The method (post-conditioning) can be applied in other clinical situations, for example, following organ transplantation when the donor organ has suffered temporary ischemia, renal angioplasty, and ablation of cerebral or peri-cerebral thromboses. Moreover, post-conditioning can be applied in conjunction with pharmacological therapy, or mimicked by pharmacological therapy utilizing mediators of the mechanisms involved in post-conditioning. As used herein, “post-conditioning reperfusion” means the application of repeated cycles of stopping or reducing perfusion followed by resuming perfusion of an organ or tissue previously affected by ischemia. As used herein, “perfusion” and “perfusing” mean blood flow to, through or within an organ or tissue. As used herein, “reperfusion” is the restoration or resumption of blood flow to, through or within an organ or tissue after a period of interruption of blood flow to, through or within the organ or tissue.

As used herein, “injury” means damage or potential damage or dysfunction of an organ or tissue, as evidenced by, for example, edema (swelling), loss of function and/or infiltration of the organ or tissue by leukocytes. An injury can be as minimal, for example, as barely perceptible swelling of the cells comprising the organ or tissue. Further, an injury can include damage to an organ or tissue that occurs during and/or after a period of ischemia (an ischemic event) or after a period of reperfusion (reperfusion injury). As used herein, an “injured” or “target” organ or tissue is an organ or tissue that has had or may have some potential damage from ischemia or reperfusion. A “leukocyte” can be a neutrophil, lymphocyte, monocyte, macrophage, basophil or eosinophil. As used herein, “ischemia” means an interrupted supply of blood to an organ or tissue that can be caused by, for example, a mechanical obstruction (i.e., a thrombus or embolus) in an artery, external compression of an artery, iatrogenic blocking of blood flow in an artery to an organ (e.g., an organ that is to be surgically removed from one subject and subsequently transplanted into another subject), and/or hypotension (low blood pressure). Hypotension can result from a cardiac arrhythmia, a neurogenic reflex causing vasodilation and subsequent pooling of blood in the lower extremities (e.g., a vasovagal reflex), hypovolemia (i.e., a reduced amount of intravascular fluid) caused by inadequate fluid intake by a subject or loss of blood by a subject following a traumatic wound. Thus, an “ischemic injury” means the damage or potential damage to an organ or tissue that results from the interruption of blood flow to the organ or tissue, i.e., an ischemic event. As used herein, a “reperfusion injury” is the damage or potential damage to an organ or tissue that results from the resumption of blood flow to the organ or tissue during or following an ischemic event. An “ischemic event” is an interruption of the blood supply to an organ or tissue. As used herein, a “total” ischemic event is a complete interruption of the blood supply to an organ or tissue. As used herein, a “subtotal” ischemic event is an incomplete interruption of the blood supply to an organ or tissue. Examples of an organ or tissue that can be subject to an ischemic event and/or suffer an ischemic injury include, but are not limited to, heart, brain, kidney, intestine, pancreas, liver, lung and skeletal muscle.

Thus, provided is a method of preventing injury to an organ or tissue in a subject during or after reperfusion following an ischemic event to the organ or tissue, comprising: a) stopping perfusion of the organ or tissue for from about 5 seconds to about 5 minutes; b) resuming perfusion of the organ or tissue for from about 5 seconds to about 5 minutes; c) repeating steps a) and b) sequentially for from about 2 to about 50 times; and d) ending stopping perfusion of the organ or tissue, thereby preventing injury to the organ or tissue in the subject during or after reperfusion following an ischemic event.

Also provided herein is a method of preventing injury to an organ or tissue in a subject during or after reperfusion following an ischemic event to the organ or tissue, comprising: a) reducing perfusion of the organ or tissue for from about 5 seconds to about 5 minutes; b) resuming perfusion of the organ or tissue for from about 5 seconds to about 5 minutes; c) repeating steps a) and b) sequentially for from about 2 to about 50 times; and d) ending reducing perfusion of the organ or tissue, thereby preventing injury to the organ or tissue in the subject during or after reperfusion following an ischemic event. As used herein, “reducing perfusion” means reducing the amount of perfusion such that injury to the organ or tissue is prevented. For example, reducing perfusion to about 20%, 15%, 10% or 5% of the expected blood flow is contemplated. Also contemplated is a combination of stopping and reducing perfusion in a single procedure.

As used herein, a subject can include domesticated animals, such as cats, dogs, etc., livestock (e.g., cattle, horses, pigs, sheep, goats, etc.), laboratory animals (e.g., mouse, rabbit, rat, guinea pig, etc.) and birds. Preferably, the subject is a mammal such as a primate, and, more preferably, is a human.

As provided herein, after reperfusion has been established, injury to an organ or tissue undergoing ischemia can be prevented by repeatedly stopping or reducing perfusion of the organ or tissue and then resuming perfusion of the organ or tissue. A cycle of stopping or reducing perfusion and resuming perfusion can be repeated for from about two to about 50 times. Stopping or reducing perfusion of the organ or tissue can last for from about 5 seconds to about 5 minutes, followed by resumption of perfusion of the organ or tissue that lasts for from about 5 seconds to about 5 minutes. After the last cycle of stopping or reducing and starting perfusion, blood flow to the organ or tissue is restored unabated, or can be under some degree of control. For example, after the last on-off cycle, blood flow can be started slowly and gradually increased until normal blood flow is achieved. A person of skill can use algorithms known in the art to determine the rate at which blood flow can be resumed.

A person of skill can stop or reduce perfusion of an organ or tissue by introducing into the lumen of a blood vessel that supplies blood to the organ or tissue a mechanical device that can be used to temporarily block blood flow in the vessel. After a selected period of time, the device can be manipulated to restore perfusion of the organ or tissue. After performing a selected number of cycles of stopping or reducing perfusion and resuming perfusion of the organ or tissue, a person of skill can remove the device from the lumen of the blood vessel so that reperfusion (i.e., blood flow to the organ or tissue) is restored. The blood vessel can be an artery or a vein, preferably an artery.

An example of a mechanical device that can be used in post-conditioning reperfusion is a catheter to which is attached a medical balloon that can be inflated within the lumen of a vessel to block blood flow to the injured organ or tissue and deflated to restore blood flow to the injured organ or tissue. A catheter/balloon device can be introduced into a blood vessel of a subject either percutaneously or directly into a vessel during an operative procedure. After the catheter/balloon is within a vessel lumen, a person of skill can guide it to a specific artery under radiologic control according to well known methods.

In another embodiment of the invention, a hollow catheter can be introduced into a vessel of a subject. The diameter of the lumen of the catheter can be large enough to permit blood, fluid or a blood/fluid combination to flow through it to the targeted organ or tissue. The catheter can be attached to a pump that is external to the subject. The pump can be activated to pump blood through the catheter to the targeted organ or tissue and inactivated to stop or reduce blood flow to the targeted organ or tissue. After reperfusion of an organ or tissue that has suffered an ischemic injury has been established, a person of skill can inactivate the pump to stop or reduce perfusion of the targeted organ or tissue. After a selected period of time, for example, from about 5 seconds to about 5 minutes, a person of skill can activate the pump to begin perfusion of the targeted organ for from about 5 seconds to about 5 minutes. The pump can be used to stop or reduce, and start perfusion of the targeted organ or tissue for from about two to about 50 cycles. After post-conditioning has been completed, the catheter can be removed from the subject.

In another embodiment of the invention, after reperfusion has been established, a medical practitioner can stop or reduce blood flow to an organ or tissue injured by ischemia, using external compression of the vessel. The practitioner can use a gloved hand, a ligature, or a surgical instrument, for example, a clamp or hemostat, to temporarily stop or reduce blood flow through the vessel to the injured organ or tissue. After blood flow through the vessel has been stopped or reduced for a selected period of time, the practitioner can remove the hand, the ligature, or the surgical instrument from the vessel, thereby removing the interruption of blood flow to the injured organ or tissue. After a selected number of cycles of temporarily stopping or reducing, and restoring perfusion of the injured organ or tissue, the practitioner can restore blood flow to the organ or tissue without further intervention.

During or after post-conditioning reperfusion of an organ or tissue previously affected by ischemia, a practitioner can administer to the subject an effective amount of a tissue protective agent in a pharmaceutically acceptable carrier that can further prevent injury to the organ or tissue. As used herein, an “effective amount” of an agent of this invention is that amount needed to achieve the desired result or results known to those skilled in the art. An example of an organ or tissue that can have the desired results of post-conditioning reperfusion is the heart, in which reduction in infarct size, decrease in myocardial edema, attenuation in release of creatine kinase, inhibition of hyperemia during early reperfusion, augmentation in endothelium-dependent vascular relaxation, decrease in neutrophil adherence to ischemic/reperfused coronary endothelium, increased contractile function and decrease in neutrophil accumulation in ischemic myocardium can be monitored and attained. Thus, a heart treated according to the method of the present invention can exhibit better overall function, for example, increased cardiac output and smaller heart size due to less severe heart failure, fewer arrhythmias and a steadier heart rate. Moreover, a subject can exhibit better tolerance to exercise and can better tolerate a subsequent heart attack.

By “pharmaceutically acceptable carrier” is meant a material that is not biologically or otherwise undesirable, i.e., the material can be administered to an individual along with the protective agent without causing substantial deleterious biological effects or interacting in a deleterious manner with any of the other components of the composition in which it is contained. The tissue protective agent can be administered through a catheter within the lumen of a vessel near the site where the vessel enters the injured organ or tissue, or can be administered intravenously or in a systemic artery. Examples of tissue protective agents include, but are not limited to, steroids to suppress localized edema and inflammation, adenosine, anti-oxidants, saline to replace lost intravascular fluid and to dilute blood, antagonists of platelet aggregation, thrombolytic agents and anticoagulants.

The dosage of the tissue protective agent will depend on the specific agent used. A person of ordinary skill in the art would know the appropriate dosage of a tissue protective agent and can vary the dosage according to the age, weight, gender and overall condition of the subject, using only routine experimentation given the teachings herein (see, e.g., Remington's Pharmaceutical Sciences, Martin, E. W. (ed.), latest edition. Mack Publishing Co., Easton, Pa.). For example, the dosage of heparin, an anticoagulant, can be from about 10 units to about 10,000 units. Other examples of tissue protective agents and their respective dosage ranges include, but are not limited to, steroids: 10 nM to 10 mM; aprotinin (serine protease inhibitor): 1U/mL−1000/mL U intracoronary); adenosine: 1 μM to 500 mM; nitric oxide and related compounds such as sodium nitroprusside: 10⁶ to 10² M; and sodium hydrogen exchange inhibitors such as cariporide: 1 nM to 100 μM.

Also provided herein is a method of preventing injury to a heart in a subject diagnosed with an ischemic event of the heart, comprising: a) clearing a lumen of a coronary artery; b) perfusing the heart for from about 5 seconds to about 5 minutes; c) stopping perfusion of the heart for from about 5 seconds to about 5 minutes; d) repeating steps b) and c) sequentially for from about 2 to about 50 times; and e) resuming perfusion of the heart, thereby preventing injury to the heart in the subject.

Further provided herein is a method of preventing injury to a heart in a subject diagnosed with an ischemic event of the heart, comprising: a) clearing a lumen of a coronary artery; b) perfusing the heart for from about 5 seconds to about 5 minutes; c) reducing perfusion of the heart for from about 5 seconds to about 5 minutes; d) repeating steps b) and c) sequentially for from about 2 to about 50 times; and e) resuming perfusion of the heart, thereby preventing injury to the heart in the subject.

As used herein, an “ischemic-reperfusion event” of a heart (heart attack) is an event that occurs when the heart muscle (myocardium) suffers an interruption in its blood supply (ischemia) that is ultimately followed by restoration of blood flow (reperfusion). During ischemia, the muscle rapidly loses function, is depleted of its energy supply and undergoes changes consistent with inflammation. A second, more robust or explosive injury occurs at the onset of reperfusion (i.e., reperfusion injury), characterized by an increase in inflammation, activation of white blood cells in the region of the heart, tissue edema and swelling, injury to the small blood vessels feeding the heart muscle in the area involved in the heart attack, and an extension of necrosis (cell death) to include greater amounts of heart tissue. By “myocardial infarction” is meant an ischemic injury to the heart in which part of the myocardium has undergone necrosis or apoptosis, i.e., programmed cell death. Therefore, injury to the heart during a heart attack occurs during both ischemia and reperfusion.

An evolving heart attack reflects the dynamic nature of injury during both ischemia and reperfusion. Thus, the injury that started or was triggered by ischemia can continue after the onset of reperfusion in which cell function may further deteriorate, and the amount of muscle actually going on to die increases with reperfusion. There is a clear relationship between ischemic injury and reperfusion injury in that the ischemic event sets the stage for reperfusion injury. The more severe the ischemic event is, the more severe the subsequent reperfusion injury is. Hence, the two events are often referred to as ischemia-reperfusion injury to reflect this intimate link between two separate but interrelated events. Interventions can be directed to either a decrease in ischemic injury or a decrease in reperfusion injury.

It is contemplated that a subject who presents to a medical facility with signs and symptoms of a heart attack can be diagnosed in time to be treated according to the methods taught herein. If during angiographic examination of the subject's coronary arteries it is determined that a coronary artery is blocked (partially or totally) by a thrombus, embolus, cholesterol plaque or other obstruction and that the blocked artery can be opened by PTCA, the practitioner can insert a balloon catheter percutaneously into a femoral vein of the subject and guide the catheter into the blocked coronary artery. After the balloon is properly localized at or near the site of blockage of blood flow in the coronary artery, the practitioner can manipulate and/or inflate the balloon to compress the thrombus, embolus, cholesterol plaque or other obstruction against the vessel wall, thereby clearing the lumen and reperfusing the myocardium.

To prevent injury and/or subsequent injury to the injured myocardium after reperfusion has been established, post-conditioning can be performed. Specifically, the practitioner can leave the balloon catheter in place and re-inflate the balloon for from about 5 seconds to about 5 minutes to stop or reduce perfusion of the injured myocardium. After the selected time period of stopped or reduced perfusion, the practitioner can deflate the balloon to restore perfusion of the myocardium for from about 5 seconds to about 5 minutes. This cycle of inflating and deflating the balloon within the lumen of the coronary artery can, for example, be repeated for from about 2 to about 50 times. After the final deflation of the balloon, the practitioner removes the balloon catheter.

In another embodiment of the invention, a subject diagnosed with an ischemic event and found to have coronary artery disease not amenable to PTCA can be treated with CABG surgery. During the operative procedure and after the diseased coronary artery has been bypassed to restore blood flow to the myocardium, a surgeon can effect post-conditioning reperfusion by stopping or reducing perfusion of the injured myocardium by compressing the grafted vessel with a gloved hand, a ligature, or with a surgical instrument, for example, a clamp or a hemostat. Stopping or reducing perfusion can be maintained for from about 5 seconds to about 5 minutes. After the selected period of time has passed, the surgeon can remove the hand, the ligature, or the surgical instrument from the vessel, thereby restoring blood flow through the graft to the injured myocardium. Perfusing the injured myocardium can last for from about 5 seconds to about 5 minutes. The cycle of stopping or reducing perfusion, and resuming perfusion of the injured myocardium can be repeated for from about two to about 50 times. At the end of the last cycle, perfusion of the injured myocardium is maintained.

The following example is put forth to provide those of ordinary skill in the art with a complete disclosure and description of how the compositions and/or methods claimed herein are made and evaluated, and is intended to be purely exemplary of the invention and is not intended to limit the scope of what the inventors regard as the invention. Efforts have been made to ensure accuracy with respect to numbers (e.g., amounts, temperature, etc.), but some errors and deviations should be accounted for. The present invention is more particularly described in the following example which is intended as illustrative only since numerous modifications and variations therein will be apparent to those skilled in the art.

### EXAMPLE

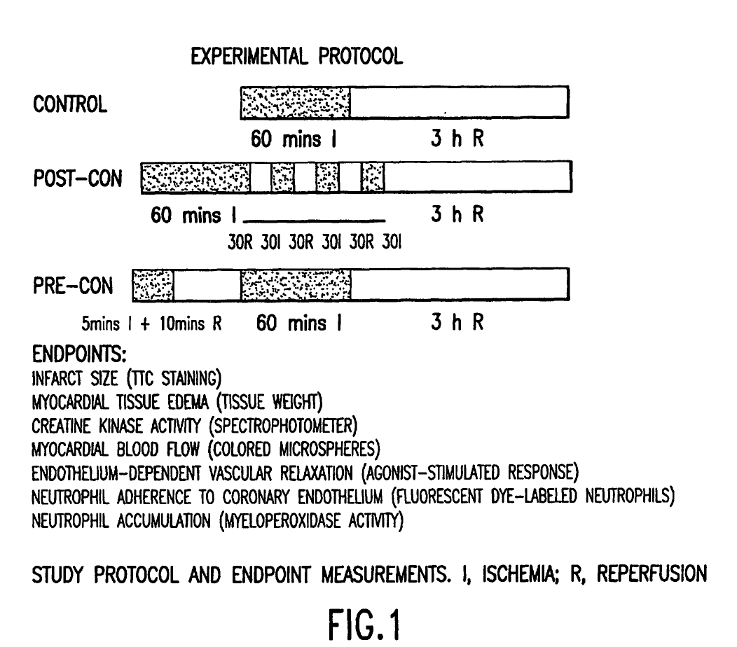

The concept of post-conditioning was tested in an opened-chest canine model of regional myocardial ischemia and reperfusion. All animals were randomly assigned to one of the following three groups (FIG. 1): 1) Control: the left anterior descending coronary artery (LAD) was reversibly occluded for 60 minutes, and the ischemic myocardium was then reperfused for 3 hours; 2) ischemic post-conditioning (Post-con): after 60 minutes of LAD occlusion, the ischemic myocardium was initially reperfused using 3 cycles of repetitively applied reperfusion followed by ischemia, i.e., 30 seconds of reperfusion followed by 30 seconds of occlusion repeated in 3 successive cycles; 3) ischemic preconditioning (Pre-con): 5 minutes of LAD occlusion and 10 minutes of reperfusion were performed before the 60 minutes of myocardial ischemia.

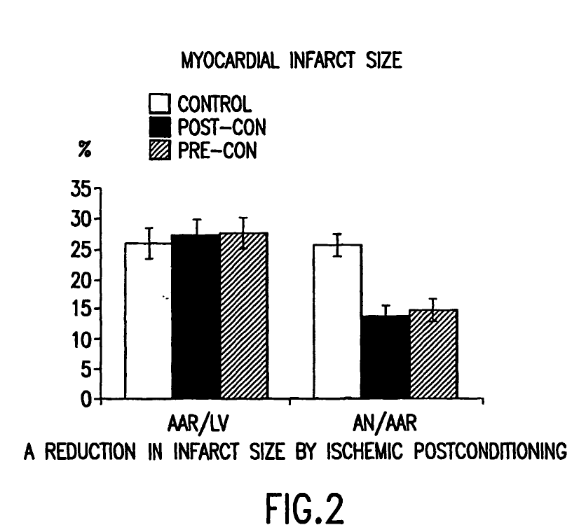

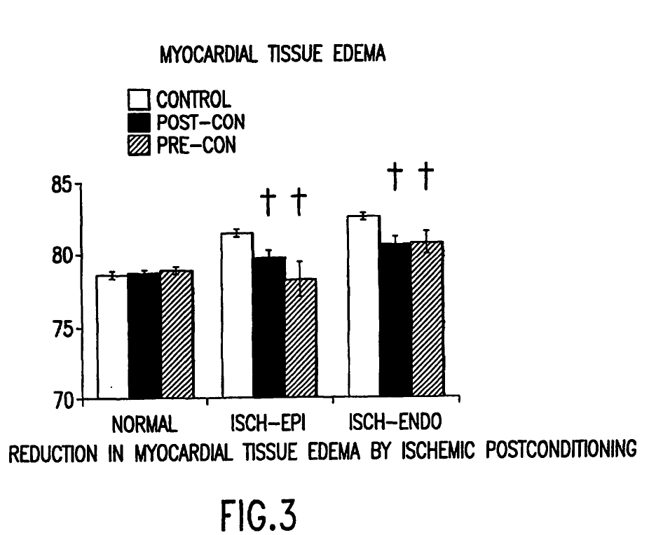

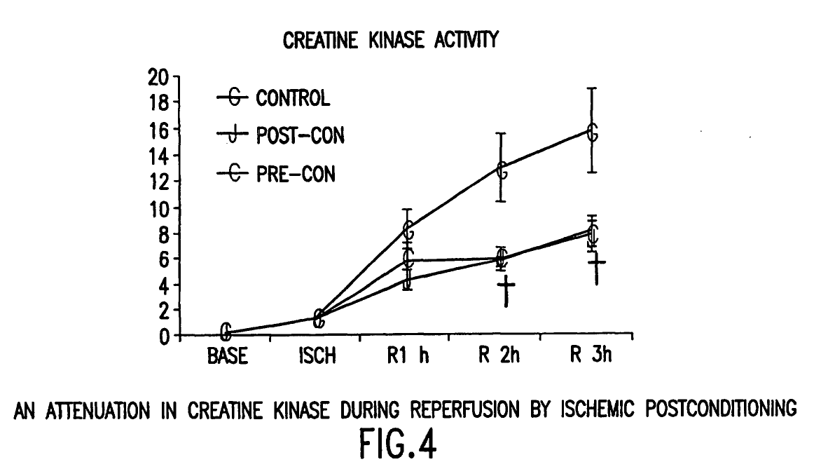

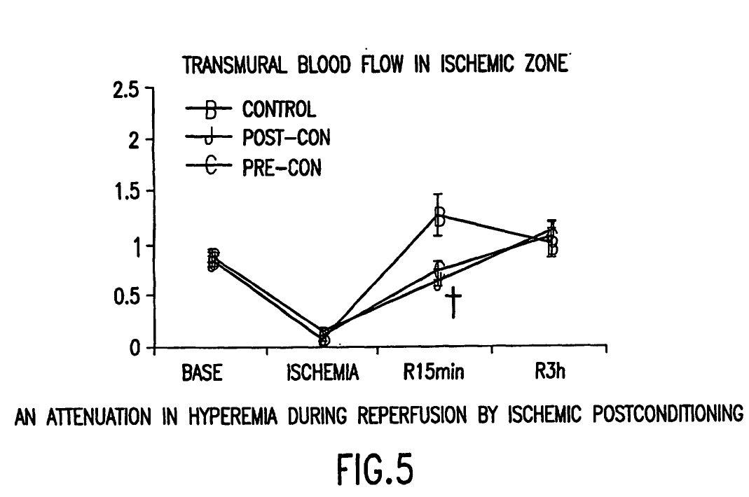

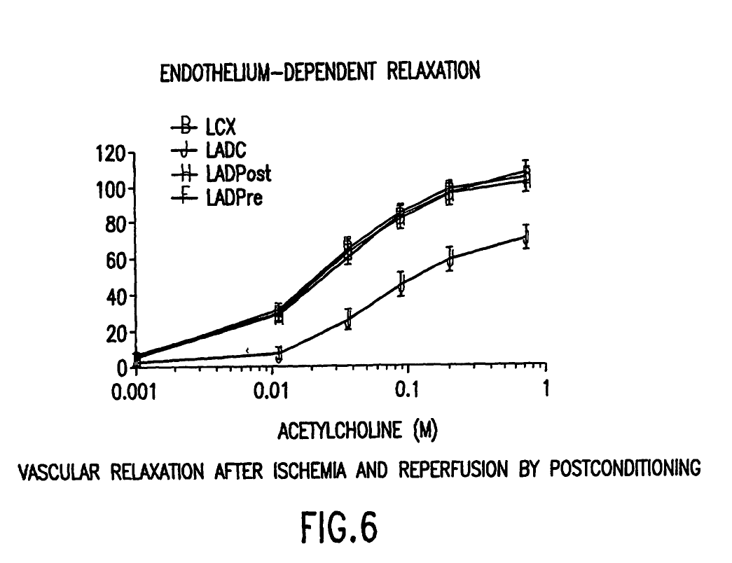

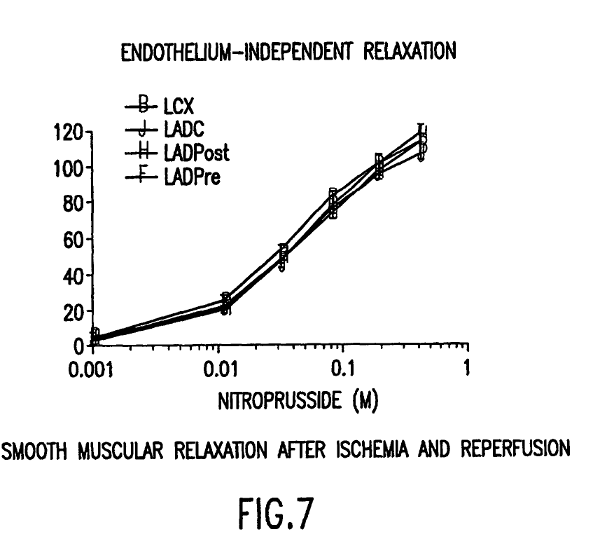

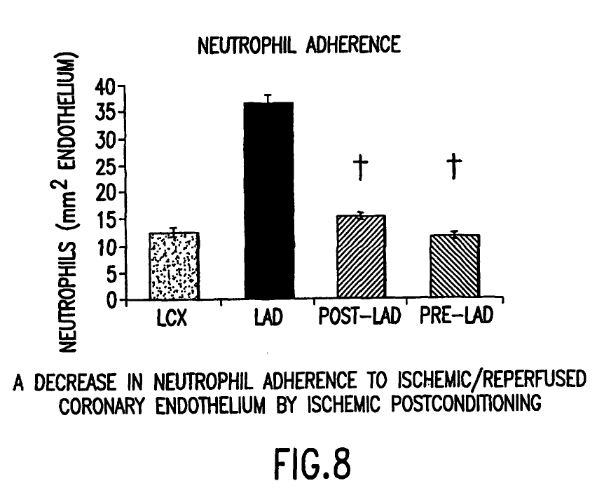

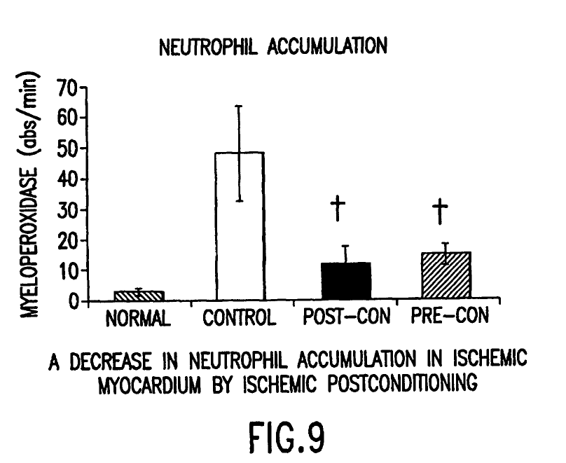

FIGS. 1-9 show the salutary effects of post-conditioning on the ischemic/reperfused heart. Those effects include reduction in infarct size measured by a vital stain (triphenyltetrazolium chloride) post-mortem [6], which was confirmed by a decrease in the release of creatine kinase measured spectrophotometrically from arterial blood [6]. Creatine kinase is an intracellular macromolecule which escapes from a cell only when there is severe, lethal injury to that cell. Moreover, post-conditioning is associated with a decrease in myocardial edema in the previously ischemic myocardium, as measured by tissue dessication. Tissue edema (water gain) occurs when the microvasculature is severely injured and fails to retain blood fluids in the vascular space. Fluid that has leaked into the myocardium can surround and compress those injured capillaries, further reducing blood flow to the heart muscle. This vascular injury has been associated with irreversible injury to the myocardium, e.g., necrosis.

Post-conditioning also inhibits post-ischemic hyperemia during early reperfusion as measured by an electronic blood flow probe placed around the target coronary artery, suggesting that there is sufficient oxygen delivery during those brief periods of intermittent perfusion to satisfy myocardial energy demands.

Post-conditioning is associated with a significantly greater endothelium-dependent vascular relaxation response to acetylcholine, as measured by in vitro techniques. Acetylcholine is an endothelial-specific stimulator of the vasorelaxant agent, nitric oxide [7]. The endothelium of coronary arteries, arterioles and venules is extraordinarily sensitive to reperfusion injury and undergoes obliteration within the first few moments of reperfusion. Salvage of the vascular endothelium is important because a healthy endothelium prevents abnormalities in blood flow regulation, thereby preventing triggering migration of neutrophils into the previously ischemic zone and the formation of blood clots in the artery. Blood clots in the reperfused vessels can cause a secondary ischemia and can ultimately lead to death of the heart tissue. The decrease in neutrophil adherence to ischemic/reperfused coronary endothelium, measured by fluorescence microscopy, also represents improvement in post-ischemic endothelial function with post-conditioning.

Further, post-conditioning attenuated neutrophil accumulation in ischemic myocardium, as measured by the myeloperoxidase (MPO) assay of tissue samples from the post-reperfusion myocardium. This suggests that post-conditioning reduced the inflammatory response to ischemia/reperfusion which has been associated with the pathogenesis of infarction, contractile dysfunction and apoptosis.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and example be considered as exemplary only, with a true scope and spirit of the invention being indicated by the following claims.

Throughout this application, various publications are referenced. The disclosures of these publications in their entireties are incorporated herein by reference into this application in order to more fully describe the state of the art to which this invention pertains.

### REFERENCES

(1) Murry C E, Jennings R B, Reimer K A. Editorial Comment: New insights into potential mechanisms of ischemic preconditioning. Circulation 1991; 84:443445.

(2) Murry C E, Jennings R B, Reimer K A. Preconditioning with ischemia: a delay of lethal cell injury in ischemic myocardium. Circulation 1986; 74:1124-1136.

(3) Murry C E, Richard V J, Reimer K A, Jennings R B. Ischemic preconditioning slows energy metabolism and delays ultrastructural damage during sustained ischemic episode. Circ Res 1990; 66:913-931.

(4) Reimer K A, Murry C E, Yamasawa I, Hill M L, Jennings R B. Four brief periods of myocardial ischemia cause no cumulative ATP loss or necrosis. Am J Physiol 1986; 251:H1306-H1315.

(5) Downey J M. Ischemia preconditioning. Nature's own cardioprotective intervention. Trends Cardiovasc Med 1992; 2:170-176.

(6) Zhao Z-Q, Nakamura M., Wang N-P, Velez D A, Hewan-Lowe K. O, Guyton R A, Vinten-Johansen J. Dynamic progression of contractile and endothelial dysfunction and infarct extension in the late phase of reperfusion. J Surg Res 2000; 94: 133-144.

(7) Lefer A M, Ma X-L, Weyrich A, Lefer D J. Endothelial dysfunction and neutrophil adherence as critical events in the development of reperfusion injury. Agents Actions Suppl 1993; 41: 127-135.

What is claimed is:

1. A method of preventing injury to an organ or tissue in a subject during or after reperfusion following an ischemic event to the organ or tissue, comprising the following steps:
a) stopping perfusion of the organ for from about 5 seconds to about 5 minutes;
b) resuming perfusion of the organ for from about 5 seconds to about 5 minutes;
c) repeating steps a) and b) sequentially for from about 2 to about 50 times; and
d) allowing uninterrupted perfusion of the organ or tissue, thereby preventing injury to the organ or tissue in the subject following an ischemic event.

2. The method of claim 1, wherein the organ or tissue is heart, brain, kidney, intestine, pancreas, liver, lung or skeletal muscle.

3. The method of claim 1, wherein the subject is a mammal.

4. The method of claim 3, wherein the mammal is a human.

5. The method of claim 1, wherein stopping perfusion is effected by a balloon within a lumen of a blood vessel that supplies blood to the organ or tissue.

6. The method of claim 5, wherein the balloon is inflatable and deflatable.

7. The method of claim 1, wherein stopping perfusion is effected by external compression of a blood vessel that supplies blood to the organ or tissue.

8. The method of claim 1, further comprising administering to the subject an effective amount of a tissue protective agent in a pharmaceutically acceptable carrier.

9. A method of preventing injury to a heart in a subject diagnosed with an ischemic event of the heart, comprising:
a) clearing a lumen of a coronary artery;
b) perfusing the heart for from about 5 seconds to about 5 minutes;
c) stopping perfusion of the heart for from about 5 seconds to about 5 minutes;
d) repeating steps b) and c) sequentially for from about 2 to about 50 times; and
e) allowing uninterrupted perfusion of the heart, thereby preventing injury to the heart in the subject.

10. The method of claim 9, wherein stopping perfusion is effected by a balloon within a lumen of the coronary artery.

11. The method of claim 10, wherein the balloon is inflatable and deflatable.

12. The method of claim 9, wherein stopping perfusion is effected by external compression of the coronary artery.

13. The method of claim 9, further comprising administering to the subject an effective amount of a tissue protective agent in a pharmaceutically acceptable carrier.

14. A method of preventing injury to an organ or tissue in a subject during or after reperfusion following an ischemic event to the organ or tissue, comprising the following steps:
a) reducing perfusion of the organ for from about 5 seconds to about 5 minutes;
b) resuming perfusion of the organ for from about 5 seconds to about 5 minutes;
c) repeating steps a) and b) sequentially for from about 2 to about 50 times; and
d) allowing uninterrupted perfusion of the organ or tissue, thereby preventing injury to the organ or tissue in the subject following an ischemic event.

15. The method of claim 14, wherein the organ or tissue is heart, brain, kidney, intestine, pancreas, liver, lung or skeletal muscle.

16. The method of claim 14, wherein the subject is a mammal.

17. The method of claim 16, wherein the mammal is a human.

18. The method of claim 14, wherein reducing perfusion is effected by a balloon within a lumen of a blood vessel that supplies blood to the organ or tissue.

19. The method of claim 18, wherein the balloon is inflatable and deflatable.

20. The method of claim 14, wherein reducing perfusion is effected by external compression of a blood vessel that supplies blood to the organ or tissue.

21. The method of claim 14, further comprising administering to the subject an effective amount of a tissue protective agent in a pharmaceutically acceptable carrier.

22. A method of preventing injury to a heart in a subject diagnosed with an ischemic event of the heart, comprising:
a) clearing a lumen of a coronary artery;
b) perfusing the heart for from about 5 seconds to about 5 minutes;
c) reducing perfusion of the heart for from about 5 seconds to about 5 minutes;
d) repeating steps b) and c) sequentially for from about 2 to about 50 times; and
e) allowing uninterrupted perfusion of the heart, thereby preventing injury to the heart in the subject.

23. The method of claim 22, wherein reducing perfusion is effected by a balloon within a lumen of the coronary artery.

24. The method of claim 23, wherein the balloon is inflatable and deflatable.

25. The method of claim 22, wherein reducing perfusion is effected by external compression of the coronary artery.

26. The method of claim 22, further comprising administering to the subject an effective amount of a tissue protective agent in a pharmaceutically acceptable carrier.
